(19) United States
(12) Patent Application Publication
(10) Pub. No.: US 2023/0172494 A1
(43) Pub. Date: Jun. 8, 2023

(54) APPARATUS, SYSTEM AND METHOD FOR MEASUREMENT OF DIABETES FACTORS

(71) Applicant: EMM Diagnostics Pte Ltd, Singapore (SG)

(72) Inventor: Cher Ming Tan, Singapore (SG)

(73) Assignee: EMM Diagnostics Pte Ltd, Singapore (SG)

(21) Appl. No.: 17/542,395

(22) Filed: Dec. 4, 2021

(51) Int. Cl. A61B 5/145; A61B 5/00

(52) U.S. Cl. CPC A61B 5/14532; A61B 5/0002; A61B 5/742; A61B 5/14507

(57) ABSTRACT

The invention is to provide a protein measurement apparatus and operation method thereof, including a host device, a display device, and a sample preparation device, wherein the host device includes a power supply, an electromagnetic-wave generator, an electromagnetic-wave detection circuit, a micro-controller and a display device. The operation method of the protein measurement apparatus includes starting the micro-electromagnetic-wave generator to generate an electromagnetic-wave signal and transmit the electromagnetic-wave signal to the sample preparation device; collecting the electromagnetic-wave reflection signal, which is reflected from the sample preparation device; detecting and analyzing the electromagnetic-wave reflection signal by using the electromagnetic-wave detection circuit; and comparing and analyzing the above-mentioned electromagnetic-wave reflection signal with characteristic curve by the microcontroller, so that the insulin concentration of a tester's saliva can be obtained.

### BACKGROUND OF THE INVENTION

### Field of Invention

The present invention relates to an apparatus, system and method for measurement of diabetes factors, particularly to an apparatus, system and method for measurement of diabetes factors, such as insulin concentration and/or glucose concentration, by using a miniature electromagnetic-wave generator.

### Description of Related Art

Insulin concentration and/or glucose concentration are closely related to the chronic metabolic disorder of human body. When insulin concentration and/or glucose concentration are abnormal, it may cause chronic metabolic diseases of human body, for example, diabetes. Two main causes of diabetes are that the pancreas of human body cannot produce enough insulin protein hormones, or human cells in the body cannot use it adequately, such as human cells are resistant to the action of insulin protein hormone. Medical community believes that due to the interaction of genetic, environmental, chemical toxins, and microorganisms, the pancreas secretes insufficient insulin in the body, or the body is resistant to the action of insulin, which reduces the ability to utilize glucose or even completely unavailable, resulting in high blood glucose, glucose in urine, and abnormal metabolism of protein and fat in the human body.

Diabetes is a chronic disease that cannot be cured. Once suffering from diabetes, patients themselves must actively pay attention to blood glucose and/or insulin control throughout their lives, so as to avoid diabetes threatening their lives, and avoid serious complications caused by diabetes. Even for healthy people, they need to pay attention to blood glucose and/or insulin of themselves from the perspective of preventive medicine. If the level of insulin and/or glucose in the blood remains very low or very high for long periods of time, it could cause hypoglycemia or hyperglycemia, respectively, leading to severe medical conditions and serious complications caused by diabetes, including tissue damage, cardiovascular disease, heart attacks, stroke, coma, retinopathy, blindness, neuropathy, kidney failure, blindness, and limb amputation, among others, and finally, death if left untreated.

According to the World Health Organization (WHO), the number of diabetes patients worldwide has increased rapidly, the number of people with diabetes rose from 108 million in 1980 to 422 million in 2014. In 2014, 8.5% of adults aged 18 years and older had diabetes. In 2019, diabetes was the direct cause of 1.5 million deaths. The International Diabetes Federation (IDF) Diabetes Atlas Ninth edition 2019 further provides information and projections on diabetes worldwide that in 2019, approximately 463 million adults (aged 20-79 years) were living with diabetes, and by 2045 this number will rise to 700 million.

Due to the rapid increase in the number of diabetes patients and its complications, it has caused a financial burden to the society, and the quality of life of patients has dropped seriously. Therefore, human body centric communication and sensing devices and applications for health care and preventive medicine has got more interest than before and led to a huge development over the last decade.

It is very inconvenient and affects the quality of life of people seriously to pay attention to glucose control and/or insulin control in their daily lives actively and continuously. Therefore, a convenient, accurate, easy-to-use, portable, inexpensive, and noninvasive diagnostic tool for diabetes is highly demanded. Such a convenient, accurate, low cost, and portable monitoring apparatus and method, especially a noninvasive detection apparatus and method, provide to diabetes patients to actively monitor the insulin status in them continuously and as early as possible in daily life, can provide early diagnosis of insulin abnormalities and early diabetes accordingly. Consequently, the medical expenditures will be reduced efficiently, and the people will be benefited as well.

### SUMMARY OF THE INVENTION

As described above, there are at least two main factors, insulin concentration and glucose concentration, which can be used as indexes for detecting diabetes.

According to the aforementioned requirements, in one perspective, the present invention provides an apparatus for measurement of diabetes factors, the apparatus comprising: a host device including a miniature electromagnetic-wave generator; a display device; and a sample preparation device. The host device is electrically or wirelessly coupled to the display device and the sample preparation device, and the miniature electromagnetic-wave generator is used for generating an electromagnetic-wave signal to scan a prepared saliva sample obtained from the sample preparation device.

In one embodiment, the host device further comprises: a power supply for supplying power to the host device; the miniature electromagnetic-wave generator electrically coupled to the power supply, for generating the electromagnetic-wave signal and transmitting the electromagnetic-wave signal to perform the scanning function on the test sample; an electromagnetic-wave detection circuit electrically coupled to the miniature electromagnetic-wave generator, for detecting a reflection electromagnetic-wave signal from the test sample; and a microcontroller electrically coupled to the electromagnetic-wave detection circuit for calculating and analyzing the reflection electromagnetic-wave signal.

In one embodiment, the electromagnetic-wave signal generated by the miniature electromagnetic-wave generator is a millimeter wave, with frequency band in the range of 1-300 GHz.

In one embodiment, the electromagnetic-wave signal generated by the miniature electromagnetic-wave generator is preferred a millimeter wave with frequency 60 GHz.

In one embodiment, the miniature electromagnetic-wave generator is installed on a chip of integrated circuits, or embedded in a packaged chip of integrated circuits.

In one embodiment, the miniature electromagnetic-wave generator is very small size, which is suitably applied to the field of 3C (Computer, Communications, Consumer-Electronics) home appliances, the field of communications devices, including smart phones, and the field of Internet of Things (IoT) and Artificial Intelligence (AI).

In one embodiment, the display device includes complete graphics display systems, portable electronics, laptop and desktop computers, television/video systems, liquid crystal displays, subtractive displays, plasma panel displays, electro-luminescence (EL) displays, electrophoretic displays, field emitter displays, discrete light emitting diode displays, organic light emitting diodes (OLEDs) displays, projectors, cathode ray tube (CRT) displays, and combinations comprising at least one of the foregoing displays.

In one perspective, the present invention provides a method for measurement of diabetes factors, the method at least comprising the steps of: providing a characteristic curve; providing a test sample on the sample preparation device; starting the miniature electromagnetic-wave generator to generating the electromagnetic-wave signal; sending the electromagnetic-wave signal from the miniature electromagnetic-wave generator to the test sample of a tester as a scanning signal to scan the test sample; collecting the electromagnetic-wave signal reflected by the test sample after scanning the test sample to form a reflection electromagnetic-wave signal; detecting the reflection electromagnetic-wave signal reflected from the test sample by the electromagnetic-wave detection circuit; confirming that the reflection electromagnetic-wave signal corresponds to the test sample of the tester's insulin concentration by the microcontroller; comparing the reflection electromagnetic-wave signal of the test sample of the tester with the characteristic curve for calculating an diabetes factor result corresponding to the test sample of the tester; and outputting and displaying the diabetes factor result of the test sample of the tester by the display device.

In one embodiment, the test sample is a human body fluid, including a saliva sample, sweat sample or tear sample.

In one embodiment, the reflection electromagnetic-wave signal of the test sample of the tester is scattered and reflected from the test sample of the tester, and then the reflection electromagnetic-wave signal is detected, received, and collected by the electromagnetic-wave detection circuit. Next, the reflection electromagnetic-wave signal is compared with the characteristic curve for calculating a diabetes factor result corresponding to the test sample of the tester.

In one perspective, the present invention provides a system for measurement of diabetes factors, the system comprising: an apparatus for measurement of diabetes factors comprising a host device including a miniature electromagnetic-wave generator; and a sample preparation device, wherein the host device is electrically or wirelessly coupled to the display device and the sample preparation device, and the miniature electromagnetic-wave generator is used for generating an electromagnetic-wave signal to scan a prepared saliva sample obtained from the sample preparation device; and a communication device wirelessly connected with the apparatus for measurement of diabetes factors.

In one embodiment, the host device further comprises: a power supply for supplying power to the host device; the miniature electromagnetic-wave generator electrically coupled to the power supply, for generating the electromagnetic-wave signal and transmitting the electromagnetic-wave signal to perform the scanning function on the test sample; an electromagnetic-wave detection circuit electrically coupled to the miniature electromagnetic-wave generator, for detecting a reflection electromagnetic-wave signal from the test sample; and a microcontroller electrically coupled to the electromagnetic-wave detection circuit for calculating and analyzing the reflection electromagnetic-wave signal.

In one embodiment, the miniature electromagnetic-wave generator includes transmitters, the electromagnetic-wave detection circuit includes receivers, and the miniature electromagnetic-wave generator and the electromagnetic-wave detection circuit are arranged in a matrix pattern and enclosed in a compact hardware transceiver chip together.

In one embodiment, the system for measurement of diabetes factors further comprising a network server wirelessly connected with the apparatus for measurement of diabetes factors and communication device respectively.

In one embodiment, the test sample is a human body fluid, including a saliva sample, sweat sample or tear sample.

In one embodiment, the apparatus for measurement of diabetes factors further comprises a display device electrically or wirelessly coupled to the host device set integrally within the host device or outside of the host device; or display device wirelessly connected with the host device set on the communication device outside of the host device.

In one embodiment, the diabetes factor result corresponding to the test sample of the tester is displayed on the display device. According to the diabetes factor result shown on the display device, people can assist in determining whether have hyperinsulinemia (ie, insulin resistance symptom or pre-diabetes symptom), which shows a possibility that testers have diabetes.

In one embodiment, from the perspective of preventive medicine, the present invention can provide a simple apparatus for measurement of diabetes factors and method to help people monitor the insulin concentration themselves at anytime and anywhere for determining whether have hyperinsulinemia (ie, insulin resistance symptom or pre-diabetes symptom), so that build up good and healthy personal life habits.

In one embodiment, the present invention provides a simple apparatus for measurement of diabetes factors and method to help the diabetic patients actively notice and warn themselves based on their insulin concentration, adjust their daily routine and diet, control their normal life and build good personal life and diet habits so as to avoid exacerbating their diabetes.

In one embodiment, the present invention provides a noninvasive apparatus for measurement of diabetes factors, which uses a miniature electromagnetic-wave generator and an electromagnetic-wave detection circuit for noninvasively detecting and monitoring protein concentration levels of human body or patients with diabetes. When insulin concentration shows an increasing trend, it may be a sign of diabetes symptom, the patients can remind themselves and take necessary actions to prevent exacerbating their diabetes.

In one embodiment, the present invention provides a simple apparatus for measurement of diabetes factors and method for monitoring the insulin concentration regularly, so as to help medical personnel and health care personnel understand the changes of the insulin concentration in diabetic patients' saliva and assist medical personnel to provide appropriate insulin or other measures/treatments.

Comparing with traditional enzyme-linked immunosorbent assay (ELISA) measurement apparatus and method, the present invention provides an apparatus, system and method for measurement of diabetes factors with high sensitivity and high accuracy. In addition, the present invention provides an apparatus, system and method for measurement of diabetes factors with high reproducibility for both multi-person detection and for single-person detection

More positively, the present invention provides a simple, easy-to-use, fast-detection, high accuracy and portable apparatus for measurement of diabetes factors, which can provide alert information of increasing insulin concentration in diabetic patients' saliva early and in time.

The objectives, technical details, features, and effects of the present invention will be better understood with regard to the detailed description of the embodiments below, with reference to the attached drawings.

### BRIEF DESCRIPTION OF THE DRAWINGS

FIG. 1 shows an apparatus for measurement of diabetes factors according to one embodiment of the present invention.

FIG. 2 shows a system for measurement of diabetes factors according to one embodiment of the present invention.

FIG. 3 shows a method for measurement of diabetes factors according to one embodiment of the present invention.

FIG. 4 shows a characteristic curve of diabetes factors versus electromagnetic-wave reflection signal based on a standard saliva sample after operating the apparatus for measurement of diabetes factors according to one embodiment of the present invention.

FIG. 5 shows actual insulin concentration values comparing with present measured insulin concentration values after operating the apparatus for measurement of diabetes factors according to one embodiment of the present invention.

### DESCRIPTION OF THE PREFERRED EMBODIMENTS

Hereinafter, the embodiments of the present invention are described accompanying with drawings. Technical terms in this specification refer to the usual term explanations in the technical field. When this specification describes or defines these terms, the definitions of these terms are subject to the description or definition in this specification. Each embodiment of the present invention has one or more technical features. Under the premise of possible implementation, those skilled in the art can selectively implement some or all of the technical features in any embodiment, or selectively combine some or all of the technical features in these embodiments. In the drawings, the same component symbols represent the same components, and for clarity, the scale, size or thickness of the components may be exaggerated.

In this invention, an apparatus for measurement of diabetes factors is provided to detect diabetes factors in a noninvasive way. Here, the diabetes factors are, for example, insulin concentration levels and/or glucose concentration levels. The present apparatus for measurement of diabetes factors is a noninvasive apparatus using a miniature electromagnetic-wave generator for noninvasively detecting and monitoring protein concentration levels of human body or patients with diabetes. The concept of present apparatus for measurement of diabetes factors is from the applied signal processing approaches to identify different protein concentrations and/or glucose concentration and to correlate them to the reflected electromagnetic-wave signal readings.

In this invention, the apparatus for measurement of diabetes factors is provided to detect insulin concentration levels and/or glucose concentration levels. Insulin hormone is an important protein hormone in human body. It is secreted by β (beta) cells in pancreas, and helps in regulating the blood glucose homeostasis in the human body. It works by acting on different cells of the body such as hepatocytes, adipocytes, and muscle cells. By insulin protein's action, these cells are then able to use up the glucose that is present in the blood and thus it helps in lowering the blood glucose levels in the human body. Whenever there is an increase in the level of glucose in the body then the insulin hormone is released. Once the insulin hormone is not released in a proper amount, there can be problems in the proper functioning of the human body. Failure of the pancreas in producing a steady supply of insulin hormone will lead to diabetes where the body's cells become unable to accept glucose in the bloodstream as caloric energy due to insulin shortage and resistance. Therefore, the insulin protein is closely related to the blood glucose. Both insulin protein concentration and/or glucose concentration are important and direct factors closely related to the patients with diabetes.

Diabetes is classified into two categories: type 1 and type 2. Deficient production of insulin in the pancreas leads to diabetes type 1, characterized by the sudden drop of glucose levels. On the other hand, ineffective use of insulin leads to diabetes type 2, which is characterized by high levels of glucose. Both conditions do not have a cure, it means that both patients with diabetes type 1 and type 2 need regular insulin protein or blood glucose monitoring for the rest of their lives. For example, patients of type 1 should check up their blood glucose level and/or insulin level in accordance with the insulin intake four times a day, while type 2 patients should do the check twice a day as recommended. In addition, monitoring of blood glucose levels and/or insulin levels is currently recognized and widely used method for the diagnosis and control of diabetes. Blood glucose and/or insulin measurements are required to determine insulin dosage and to detect abnormal glucose levels and/or insulin levels indicating illnesses, dietary changes, or adverse medication responses.

In this invention, the apparatus and method for measurement of diabetes factors are provided as a kind of non-invasive measurement apparatus and method for diagnosis and control of diabetes. Comparing with the present invention, the prior blood glucose monitoring methods are usually invasive measurements, which is to drag a sample of blood from the fingertip and analyze it on a glucometer. Finger-pricking technique has been the major medically accepted daily glucose-monitoring technique for diabetic patients. It brings a lot of inconvenience in life of patients with diabetes and a sense of rejection in their mentality, making the quality of life of patients dropped seriously.

Furthermore, the present invention provides measurement apparatus and method to detect protein concentration levels and/or glucose concentration levels in saliva samples obtained from the human body or prepared in the laboratory. The present invention chooses to use protein-detecting techniques from external body fluids, such as saliva, sweat or tear, to avoid the inconvenience and risks of infection from other invasive techniques. Here, the present invention takes saliva as a test sample and as an exemplary detecting target.

As one skilled in the art knows, saliva is a complex mixture that not only contains various proteins, but also DNA (deoxyribonucleic acid), RNA (ribonucleic acid), fatty acids and various microorganisms, etc. In addition, various protein components in the blood are also present in saliva. As far as we know, there are about 1166 kinds of proteins in human saliva, and most of them can also be found in blood plasma and tear fluid. By comparing human body's salivary insulin protein and/or glucose with blood insulin protein and/or glucose, one can find the two insulin proteins and/or glucoses have the same fluctuation trend and the linear correlation between them is individual dependent, only there is a time lag between the peak insulin concentration levels and/or glucose concentration levels from blood and from saliva. Since saliva contains proteins and/or glucose corresponding to protein components and/or glucose in the blood, it can be inferred that there are a large number of diagnostic analytes present in saliva. Therefore, the protein concentration and/or glucose concentration changes in saliva can substantially reflect the changes of various protein concentration and/or glucose concentration in the blood.

In this invention, the apparatus for measurement of diabetes factors is a kind of an integrated electromagnetic-wave radar system which uses electromagnetic-wave radars transmitting to the saliva of the patients and then identifying different protein concentrations by correlating them to the reflected electromagnetic-wave signal readings from the saliva of the patients to achieve noninvasively monitoring protein concentration levels in saliva of patients with diabetes.

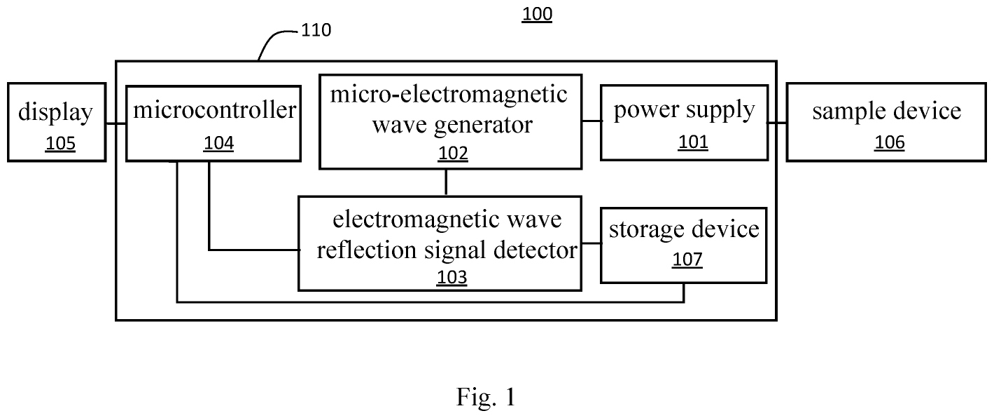

FIG. 1 shows an apparatus for measurement of diabetes factors 100 according to one embodiment of the present invention. The apparatus for measurement of diabetes factors 100 comprises a host device 110, a display device 105, and a sample preparation device 106, wherein the host device 110 is electrically or wirelessly coupled to the display device 105 and the sample preparation device 106 respectively.

Referring to FIG. 1, the host device 110 of the apparatus for measurement of diabetes factors 100 comprises a power supply 101, a miniature electromagnetic-wave generator 102, an electromagnetic-wave detection circuit 103, a microcontroller 104, and a storage device 107. The power supply 101 is electrically coupled to the miniature electromagnetic-wave generator 102 and the sample preparation device 106, for supplying power to the host device 110. The miniature electromagnetic-wave generator 102 is electrically coupled to the electromagnetic-wave detection circuit 103, for generating an electromagnetic-wave signal and scanning a saliva sample from a tester. The electromagnetic-wave detection circuit 103 is electrically coupled to the microcontroller 104, for detecting a reflection electromagnetic-wave signal. The microcontroller 104 is electrically coupled to the display device 105, for calculating and analyzing the above-mentioned reflection electromagnetic-wave signal. The storage device 107 is electrically coupled to the microcontroller 104 and the electromagnetic-wave detection circuit 103, for storing data and information.

It is noted that the present invention is a non-invasive apparatus for measurement of diabetes factors, which uses insulin protein as a sample target to detect protein concentration. Therefore, it can be seen that the detection of insulin protein is used as an example to describe the embodiments of the present invention. Here, the choice of insulin protein is based on the fact that the insulin concentration is an important factor relating to diabetes. Since insulin protein/hormone is secreted by β (beta) cells in pancreas, the functions of insulin include that insulin can participate in the regulation of carbohydrate and fat metabolism, control blood glucose balance, and promote the liver and skeletal muscle cells to convert blood glucose into glucose for use. That is, insulin can convert blood glucose into glycogen and intracellular glucose. Therefore, when the human body lacks effective insulin, it will result in high blood glucose and diabetes easily.

In one embodiment of the present invention, the apparatus for measurement of diabetes factors 100 as shown in FIG. 1 comprises the power supply 101. The power supply 101 has the function of providing power. The power supply 101 can be, but is not limited to, a regulated power supply, a AC to DC power supply or a DC to DC power supply, etc.

In one embodiment of the present invention, the apparatus for measurement of diabetes factors 100 as shown in FIG. 1 comprises the miniature electromagnetic-wave generator 102, having the function of generating an electromagnetic-wave signal, and scanning a saliva sample from a tester. That is, the miniature electromagnetic-wave generator 102 can generate an electromagnetic-wave signal and transmit the electromagnetic-wave signal to perform a scanning function on the saliva sample from the tester. Because of the small size of the miniature electromagnetic-wave generator 102, it can be installed on a variety of electric devices, even be installed on the chip of integrated circuits, and embedded in the packaged chip of integrated circuits. Therefore, the miniature electromagnetic-wave generator 102 can be adapted to lots of applications, especially adapted to be used in the field of 3C (Computer, Communications, Consumer-Electronics) home appliances, as well as in the field of communications devices, such as smart phones, and other applications, such as Internet of Things (IoT) or Artificial Intelligence (AI) applications.

In one embodiment of the present invention, the miniature electromagnetic-wave generator 102 can generate the electromagnetic-wave signal. Among different frequency ranges of the electromagnetic-wave signal, millimeter-wave (referred to as mm-wave, also known as IEEE 802.15.3c) frequencies are preferred here applied in developing the present apparatus for many reasons: such as enhanced security, reduced interference, and spectrum availability. which is preferred a millimeter wave signal. The millimeter wave is suitable for scanning the saliva sample from the tester and reflected by the saliva sample from the tester. Then the reflected wave of the saliva is detected and analyzed to get a diabetes factor result. Herein, the Millimeter Wave is a kind of electromagnetic-wave, between microwave and light wave. Its specification definition includes the wavelength range of 1-10 mm, the transmission speed of 300,000 kilometers per second, and the frequency band of 1 GHz to 300 GHz. Especially, according to the present invention, the millimeter wave generated by the miniature electromagnetic-wave generator 102 is preferred with frequency 60 GHz, which is particularly suitable used for scanning the saliva sample from the tester and reflected by the saliva sample from the tester, and then the reflected wave being detected and analyzed to get an diabetes factor result.

In one embodiment of the present invention, referring to the apparatus for measurement of diabetes factors 100 as shown in FIG. 1, the electromagnetic-wave detection circuit 103 has the function of receiving, sensing and collecting the electromagnetic-wave signal reflected from the saliva sample from the tester. That is, the electromagnetic-wave detection circuit 103 is used to detect the reflection electromagnetic-wave signal which is generated original by the miniature electromagnetic-wave generator 102 and then reflected from the saliva sample after scanning the saliva sample. Therefore, the electromagnetic-wave detection circuit 103 can receive the scanning electromagnetic-wave signal and the reflecting electromagnetic-wave signal to perform the receiving and sensing function.

In one embodiment, the miniature electromagnetic-wave generator 102 and the electromagnetic-wave detection circuit 103 of the apparatus for measurement of diabetes factors 100 are integrated to form a kind of radar system, such as a frequency-modulated continuous-wave 60 GHz radar system. The miniature electromagnetic-wave generator 102 can include, but is not limited to, transmitters. The electromagnetic-wave detection circuit 103 can include, but is not limited to, receivers. For example, the transmitters in the miniature electromagnetic-wave generator 102 and the receivers in the electromagnetic-wave detection circuit 103 can be arranged in a matrix pattern, and enclosed in a compact hardware chip together, named transceiver chip, by a semiconductor process technology.

In one embodiment, the present apparatus for measurement of diabetes factors is preferred an integrated millimeter-wave (mm-wave) radar system which uses mm-wave radars for noninvasively monitoring protein concentration levels of patients with diabetes.

In one embodiment of the present invention, the apparatus for measurement of diabetes factors 100 comprises the storage device 107, which can be random access memory storage device, including, e.g., a dynamic random access memory, or other types of non-transitory machine-readable storage devices; magnetic, magneto-optical disks, or optical disks; and CF card (CompactFlash), MMC card (Multimedia Card), mini sd (Secure Digital) memory card, micro mini sd (Secure Digital) memory card, etc.

In one embodiment, an electromagnetic-wave is generated and is modulated onto the proper frequency band, such as 1-300 GHz, and radiated periodically at high frequency, such as 1-10 KHz by the miniature electromagnetic-wave generator 102. Then with part of the transmitted energy being scattered by a target sample, a reflected electromagnetic-wave is collected by the receivers in the electromagnetic-wave detection circuit 103.

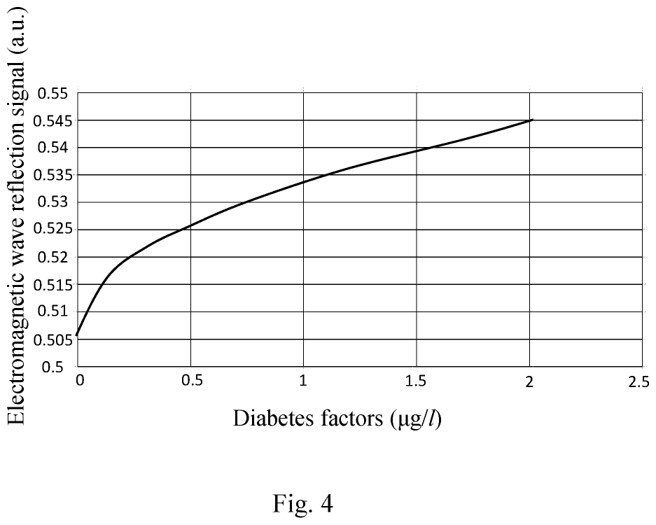

In one embodiment of the present invention, referring further to the apparatus for measurement of diabetes factors 100 as shown in FIG. 1, the microcontroller 104 is used to calculate and analyze the above-mentioned reflection electromagnetic-wave signal by comparing and analyzing the measured reflection electromagnetic-wave signal with the characteristic curve of FIG. 4. The diabetes factors, such as insulin concentration and/or glucose concentration, of the saliva sample can then be obtained according to the calculating and analyzing results of the microcontroller 104.

In one embodiment, the collected raw data in the electromagnetic-wave detection circuit 103 can be stored in the storage device 107. The collected raw data in the electromagnetic-wave detection circuit 103 can can further be analyzed in the microcontroller 104 using general signal processing techniques to identify the corresponding insulin concentrations in blood. This work is an optional step depending on other requirement or goal of modeling human blood for a continuous monitoring.

In one embodiment of the present invention, referring further to the apparatus for measurement of diabetes factors 100 as shown in FIG. 1, the display device 105 has the function of displaying measurement and analysis results of insulin concentration. The display device 105 can include, but is not limited to, complete graphics display systems, such as portable electronics, laptop and desktop computers, and television/video systems, etc. The display device 105 can also include, but is not limited to, a variety of display components, such as liquid crystal displays, subtractive displays, plasma panel displays, electro-luminescence (EL) displays, electrophoretic displays, field emitter displays, discrete light emitting diode displays, organic light emitting diodes (OLEDs) displays, projectors, cathode ray tube (CRT) displays, and the like, and combinations comprising at least one of the foregoing displays.

In one embodiment of the present invention, referring to the apparatus for measurement of diabetes factors 100 as shown in FIG. 1, the storage device 107 can be any of various memory storage devices, such as, but not limited to, random access memory (RAM) storage devices (including a dynamic random access memory, or other types of non-transitory machine-readable storage devices), magnetic disks, optical disks, magneto-optical disks, Compact Flash (CF) card, Multimedia (MMC) card, mini Secure Digital (SD) memory card, micro mini SD memory card, etc.

In one embodiment of the present invention, referring further to the apparatus for measurement of diabetes factors 100 as shown in FIG. 1, the sample preparation device 106 has the function of carrying a prepared saliva sample from a tester. In addition, the sample preparation device 106 has also the function of preparing the saliva sample, including adding a specific protein detection reagent to the saliva sample, wherein specific protein detection reagent includes an insulin protein detection reagent. In one embodiment, the insulin protein detection reagent is preferred as an above-mentioned standard saliva sample with standard insulin concentration.

In one embodiment, saliva sampling procedure can be referred from some prior literatures on saliva analysis or be developed based on the present objectives. For example, the saliva sampling procedure can include, but not limited to: rinsing a tester's mouth with water and waiting for two minutes; minimizing swallowing and holding saliva in tester's mouth; placing dental sterilized cotton sponge in tester's mouth and chewing until it is soaked with saliva; depositing sponge into syringe directly from the tester's mouth without touching it to avoid contamination; inserting plunger into the syringe; squeezing saliva through preinstalled membrane in the bottom of the syringe into sterilized tubes gently; preserving sample tubes in 4° C. chill box; taking the saliva samples on the sample preparation device 106 for detecting or measuring.

In one embodiment of the present invention, referring to the apparatus for measurement of diabetes factors 100 as shown in FIG. 1, the sample preparation device 106 is used to carry body fluid samples, such as saliva samples, sweat samples or tear samples, of the tester. Here saliva sample is preferred chosen as an example. When the miniature electromagnetic-wave generator 102 generates an electromagnetic-wave signal and transmits the electromagnetic-wave signal to the sample preparation device 106, the saliva sample from the tester carried by the sample preparation device 106 receives the electromagnetic-wave signal, is scanned by the electromagnetic-wave, and then reflects a reflection signal. The reflection signal is also an electromagnetic-wave signal. According to the present invention, the intensity and phase of the reflection electromagnetic-wave signal changes according to the dielectric constant of the saliva sample. And the dielectric constant of the saliva sample changes according to the insulin concentration therein. Since the saliva sample carried by the sample preparation device 106 includes different insulin concentration depending on different testers, the dielectric constants of the saliva samples are also different corresponding to different insulin concentrations.

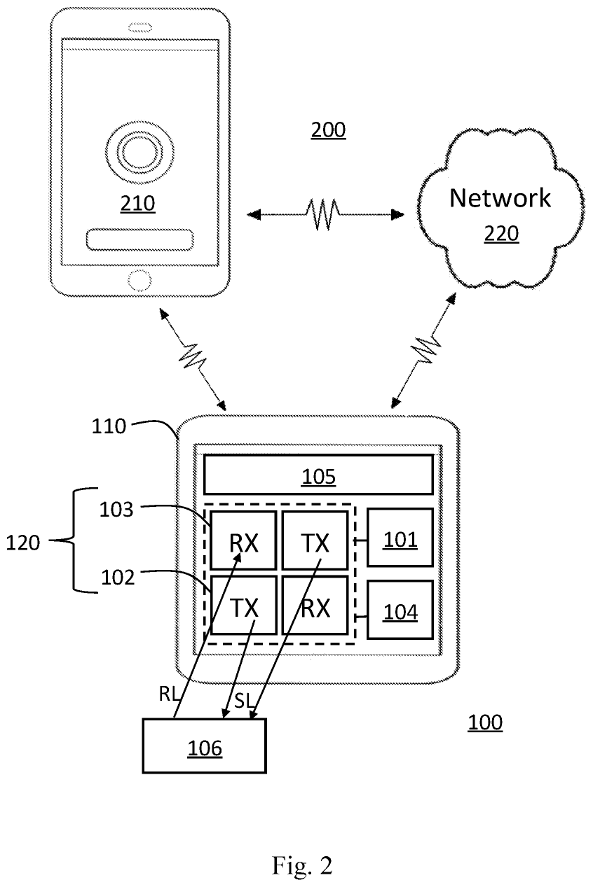

Referring to FIG. 2, it shows a system for measurement of diabetes factors according to one embodiment of the present invention. The same component symbols shown in FIG. 1, such as 100,101,102,103,104,105,106, represent the same components in FIG. 2, and for clarity, the scale, size or thickness of the components may be adjusted or exaggerated. As shown in FIG. 2, the system for measurement of diabetes factors 200 comprises a communication device 210, a network server 220, a host device 110, a display device 105, and a sample preparation device 106. The host device 110 is electrically or wirelessly coupled to the display device 105 and the sample preparation device 106 respectively. The host device 110 is wirelessly connected with the communication device 210 and network server 220 respectively. The communication device 210 is wirelessly connected with the network server 220.

In one embodiment of the present invention, referring to the system for measurement of diabetes factors 200 as shown in FIG. 2, the host device 110 further comprises a power supply 101, a miniature electromagnetic-wave generator 102, an electromagnetic-wave detection circuit 103, and a microcontroller 104. The host device 110 occupies a small volume due to the small size of the miniature electromagnetic-wave generator 102 and electromagnetic-wave detection circuit 103. Therefore, the apparatus for measurement of diabetes factors 100 can be a kind of portable device or handheld device, due to the small size of the host device 110. As shown in FIG. 2, the power supply 101 is electrically coupled to the miniature electromagnetic-wave generator 102 and the sample preparation device 106, for supplying power to the host device 110. The miniature electromagnetic-wave generator 102 is electrically coupled to the electromagnetic-wave detection circuit 103. The miniature electromagnetic-wave generator 102 can include, but is not limited to, transmitters TX, for generating an electromagnetic-wave signal SL and scanning a prepared saliva sample obtained from the sample preparation device 106. The electromagnetic-wave detection circuit 103 is electrically coupled to the microcontroller 104. The electromagnetic-wave detection circuit 103 can include, but is not limited to, receivers RX, for receiving and detecting a reflection electromagnetic-wave signal RL, which is reflected or scattered by the saliva sample obtained from the sample preparation device 106. The microcontroller 104 is electrically or wirelessly coupled to the display device 105, for calculating and analyzing the above-mentioned reflection electromagnetic-wave signal.

In one embodiment of the present invention, referring to the system for measurement of diabetes factors 200 as shown in FIG. 2, the miniature electromagnetic-wave generator 102 (eg, transmitters TX) and the electromagnetic-wave detection circuit 103 (eg, receivers RX) can be arranged in a matrix pattern, as shown in FIG. 2, and enclosed in a compact hardware chip together, named transceiver chip 120, by a semiconductor process technology.

In one embodiment of the present invention, referring to the system for measurement of diabetes factors 200 as shown in FIG. 2, the communication device 210 can be any of various mobile devices, such as a cellular phone, personal digital assistant (PDA), tablet computer, electronic reader, pocket computer, portable gaming device or any other type of mobile device capable of communicating with the host device 110 and/or the network server 220.

In one embodiment of the present invention, referring to the system for measurement of diabetes factors 200 as shown in FIG. 2, the display device 105 is electrically or wirelessly coupled to the host device 110, which can be set integrally within the host device 110. Or, as shown in FIG. 1, the display device 105 can be set outside of the host device 110. Or, the display device 105 is wirelessly connected with the host device 110 and set on the communication device 210 outside the host device. That is, the measuring and analyzing results of insulin concentration of the tester's saliva can be displayed on other devices, such as the communication device 210, instead of the display device 105, so that the display device 105 can be omitted from the apparatus for measurement of diabetes factors 100.

In one embodiment, the present invention further provides a characteristic curve, as shown in FIG. 4. which is depicted by measuring the diabetes factors (including insulin concentration and/or glucose concentration) (μg/l) of a standard saliva sample as the horizontal axis, and corresponding reflection electromagnetic-wave signal (arbitrary electromagnetic-wave unit, a.u.) as the vertical axis, by operating the apparatus for measurement of diabetes factors on a standard saliva sample, wherein the standard saliva sample has standard reference insulin concentration. Therefore, comparing with the characteristic curve of the diabetes factors and the reflection electromagnetic-wave signal based on a standard saliva sample with standard insulin concentration (as shown in FIG. 4) can obtain correct insulin concentration of the tester by measuring the dielectric constant and the intensity and phase of the reflection electromagnetic-wave signal of the saliva sample from the tester.

In one embodiment, the characteristic curve of the present invention is called a standard curve method, which establishes a standard curve from a standard sample to determine the concentration of the sample to be tested. Protein quantification requires a known standard sample to obtain a standard curve. Here, the present invention can establish a standard curve based on a standard saliva sample with known insulin concentration for use, such as the characteristic curve shown in FIG. 4. According to the diabetes factor characteristic curve, the concentration of diabetes factors (including insulin protein concentrations and/or glucose concentrations) in the saliva sample from the tester can be measured.

In one embodiment of the present invention, a set of various saliva samples of different insulin concentrations could be prepared on test tubes in laboratory and labeled accordingly. As shown in FIG. 4, for example, there are twenty saliva samples provided and carried in the sample preparation device 106 for measuring. An electromagnetic-wave from the micro-electromagnetic-wave generator 102 and is transmitted to the sample preparation device 106 for scanning the saliva samples carried by the sample preparation device 106. Then, the electromagnetic-wave is reflected from the scanned saliva samples and received by the electromagnetic-wave detection circuit 103. The insulin concentration can be accordingly obtained

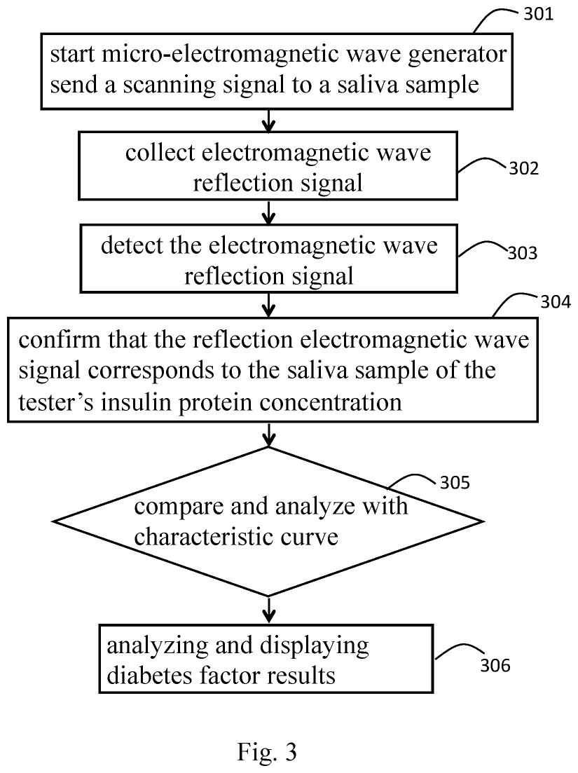

Referring to FIG. 3, an operation method of the apparatus for measurement of diabetes factors 100 is disclosed according to one embodiment of the present invention. As shown in FIG. 3, the step 301 includes starting the micro-electromagnetic-wave generator 102. The micro-electromagnetic-wave generator 102 generates an electromagnetic-wave signal and transmits the electromagnetic-wave signal to the sample preparation device 106 for scanning the saliva sample carried by the sample preparation device 106.

In one embodiment of the present invention, referring further to the operation method of the apparatus for measurement of diabetes factors 100 as shown in FIG. 3, the step 302 includes collecting the electromagnetic-wave reflection signal, which is originally generated by the miniature electromagnetic-wave generator 102 and reflected from the saliva sample carried by the sample preparation device 106 after scanning the saliva sample carried by the sample preparation device 106.

In one embodiment of the present invention, referring further to the operation method of the apparatus for measurement of diabetes factors 100 as shown in FIG. 3, the step 303 includes detecting and analyzing the electromagnetic-wave reflection signal reflecting from the saliva sample carried by the sample preparation device 106 by using the electromagnetic-wave detection circuit 103.

In one embodiment of the present invention, referring further to the operation method of the apparatus for measurement of diabetes factors 100 as shown in FIG. 3, the step 304 includes confirming that the electromagnetic-wave reflection signal corresponds to the tester's insulin concentration by the microcontroller 104.

In one embodiment of the present invention, referring further to the operation method of the apparatus for measurement of diabetes factors 100 as shown in FIG. 3, the step 305 includes comparing and analyzing the above-mentioned electromagnetic-wave reflection signal with characteristic curve by the microcontroller 104, so that the diabetes factors (including insulin concentration and/or glucose concentration) of the tester's saliva can be obtained.

In one embodiment of the present invention, referring further to the operation method of the apparatus for measurement of diabetes factors 100 as shown in FIG. 3, the step 306 includes displaying analyzing results of insulin concentration of the tester's saliva by the display device 105. By displaying the concentration of the insulin protein in the tester's saliva, it helps to determine whether the testers have diabetes. That is, by providing the information of insulin concentration in the tester's saliva, the accuracy of determination whether the testers having diabetes can according be improved.

Referring to FIG. 4, a characteristic curve of diabetes factors versus electromagnetic-wave reflection signal is illustrated based on a standard saliva sample after operating the apparatus for measurement of diabetes factors according to one embodiment of the present invention. As shown in FIG. 4, the characteristic curve of the diabetes factors after operating the apparatus for measurement of diabetes factors is depicted according to the measured reflection electromagnetic-wave signal (vertical axis) versus the corresponding insulin concentration (horizontal axis), since the intensity and phase of the reflection electromagnetic-wave signal changes according to the dielectric constant of the saliva sample, and the dielectric constant of the saliva sample changes according to the insulin concentration.

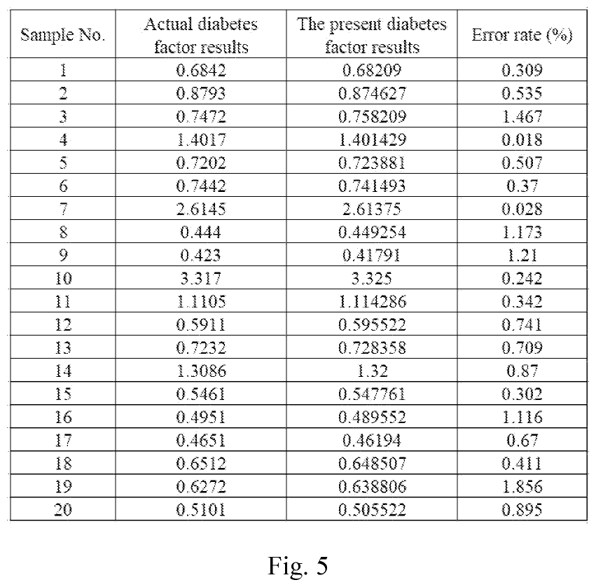

Referring to FIG. 5, actual diabetes factors values comparing with present measured diabetes factors values after performing the method for measurement of diabetes factors are listed according to one embodiment of the present invention. As shown in FIG. 5, there is disclosed a “Sample number” column, an “actual insulin concentration” column, a “present insulin concentration” column, and an “error rate” column, in sequence from left to right. As shown in FIG. 5, the most right “error rate” column shows that the error rate of the present invention is extremely low and does not exceed the error rate of 1.9%, which is far accurate beyond conventional methods and experimental data. The present invention shows excellent accuracy as compared to the conventional methods. Therefore, the apparatus and method for measurement of diabetes factors of the present invention can get extremely high accurate detection values for detecting insulin concentration.

In one embodiment of the present invention, due to the small size of the miniature electromagnetic-wave generator 102, the total capacity and volume of the apparatus for measurement of diabetes factors 100 is also very small and can be installed in the field of 3C household appliances or communication equipment easily, such as smart phones. Therefore, the present invention is suitable to be used for individual glucose monitoring at home or in daily activities. That is, the present invention can perform diabetes detection at any time, and it can monitor whether diabetes occurs at anytime and anywhere for testers. And, the present miniaturized saliva-based diagnostic apparatus enables to provide quick and reliable results for clinical decision-making and treatment out-comes-predicting.

Furthermore, in one embodiment of the present invention, the apparatus for measurement of diabetes factors provides a non-invasive apparatus for monitoring the protein of the human body. When the insulin concentration in the saliva of a normal tester increases in an increasing trend, it may be a sign of high insulin protein symptom (ie, insulin resistance symptom or pre-diabetes symptom), which shows a possibility that testers have diabetes. Therefore, the tester itself can be alerted early and in time by the apparatus for measurement of diabetes factors and take necessary actions to prevent diabetes. The present invention can also assist normal people to check their daily habits to ensure their normal living habits without becoming a diabetic patient.

According to the aforementioned embodiments of the present invention, for diabetic patients, the apparatus for measurement of diabetes factors can monitor the insulin concentration regularly, so as to help medical personnel and health care personnel understand the changes of the insulin concentration in diabetic patients' saliva and assist medical personnel to provide appropriate insulin or other measures/treatments. More positively, the apparatus for measurement of diabetes factors can provide alert information of increasing insulin concentration in their saliva early and in time, help the diabetic patients adjust their daily routine, ensure their normal life and build good personal life habits so as to avoid exacerbating their diabetes.

In summary, the present invention provides a very easy non-invasive operation method and apparatus to monitoring insulin concentration for testers. In addition to preventing normal people from suffering from diabetes, the present invention can also remind normal people to actively pay attention to blood glucose control and maintain good living habits. In other words, the present invention can help remind normal people to avoid the occurrence of diabetes, and can prevent diabetes from threatening their lives, so as to avoid affecting the quality of life of normal people.

The apparatus for measurement of diabetes factors of the present invention provides a real-time insulin concentration monitoring for diabetic patients, thus leading to better management of the diabetes, and enabling earlier diagnosis of the disease and earlier warning of adverse events such as diabetic coma, heart attacks, and strokes, etc. It is estimated that nearly half of diabetics suffer from illness without knowing it, because the symptoms of diabetes often occur slowly and are often difficult to detect at the beginning. Diabetes is the killer of the systemic vasculature. Diabetes causes lots of chronic comorbidities, including small vessel disease, such as retinopathy, kidney disease, neuropathy; and macrovascular disease, also known as arteriosclerosis. The apparatus for measurement of diabetes factors can let the diabetes condition be better controlled and managed, delay to cause tissue damage, cardiovascular disease, heart attacks, stroke, coma, retinopathy, blindness, neuropathy, kidney failure, blindness, and limb amputation by earlier diagnosis and earlier warning of the disease events.

From the perspective of preventive medicine, the present invention provides a simple detection and monitoring apparatus and method that can diagnose insulin resistance and diabetes early, so that it can avoid suffering from type 2 diabetes, and this can greatly reduce national medical expenditures and benefit the public. The present invention is developed based on the following concepts: due to insulin resistance and early diabetes patients, the insulin concentration in their blood or saliva will rise. If the changes in the insulin concentration in body fluids can be actively and correctly monitored early, the early diagnosis of hyperinsulinemia (ex: insulin resistance or pre-diabetes) can be achieved. In pre-diabetes, people can adjust their diet and improve exercise to prevent them from suffering from diabetes by continuously monitoring their insulin concentration by using the apparatus for measurement of diabetes factors and method. Even if people get diabetes, the diabetes cannot be cured since the death of their pancreatic cells and the patients must take medication throughout their lives, the non-invasive apparatus and method for measurement of diabetes factors of the present invention can still provide continuous monitoring to help patients maintain a good quality of life.

In one embodiment, the present invention takes the measurement of insulin protein as an example, since insulin is a protein hormone secreted by pancreatic β (beta) cells into the blood, and the insulin concentration can reflect the blood glucose level in the blood. That is, the insulin protein is an important and direct factor for the patients with diabetes.

In one embodiment, the present invention takes the detection of insulin concentration in body fluid, saliva, as an example. Since the correlation between the salivary insulin concentration with blood insulin concentration at fasting is constant for each person, the present invention establishes a correct noninvasive diagnosis of diabetes through saliva instead of blood. Thus, the present invention provides a novel protein detection device and method for early diagnosis, on-time treatment, continuous management by noninvasively tracking diabetes status and detecting salivary insulin concentration real-time and at any time, which improves patients' life quality very much.

In addition to detecting changes in insulin concentration in body fluids, the present invention can also be extended to detect the concentration changes of other disease-related proteins in other body fluids. In summary, the apparatus for measurement of diabetes factors of the present invention can be used for detecting or monitoring, not limited to insulin protein, but also other various proteins in saliva of human body. And, the apparatus for measurement of diabetes factors can be used for detecting or monitoring various proteins which existed not only in saliva, but also in other external body fluids, such as sweat or tear, so as to provide wide applications for non-invasive monitoring techniques. Since protein concentration is closely related to the chronic metabolic disorder of human body, the present non-invasive protein monitoring techniques also provide potentially wide applications for detecting other chronic metabolic disorder of human body.

The present invention has been described in considerable detail with reference to certain preferred embodiments thereof. It should be understood that the description is for illustrative purpose, not for limiting the scope of the present invention. An embodiment or a claim of the present invention does not need to achieve all the objectives or advantages of the present invention. The title and abstract are provided for assisting searches but not for limiting the scope of the present invention. Those skilled in this art can readily conceive variations and modifications within the spirit of the present invention.

What is claimed is:

1. A apparatus for measurement of diabetes factors comprising:
a host device including a miniature electromagnetic-wave generator;
a display device; and
a sample preparation device,
wherein the host device is electrically or wirelessly coupled to the display device and the sample preparation device, and the miniature electromagnetic-wave generator is used for generating an electromagnetic-wave signal to scan a test sample obtained from the sample preparation device.

2. The apparatus for measurement of diabetes factors of claim 1, wherein the host device further comprises:
a power supply for supplying power to the host device;
the miniature electromagnetic-wave generator electrically coupled to the power supply, for generating the electromagnetic-wave signal and transmitting the electromagnetic-wave signal to perform the scanning function on the test sample;
an electromagnetic-wave detection circuit electrically coupled to the miniature electromagnetic-wave generator, for detecting a reflection electromagnetic-wave signal from the test sample; and
a microcontroller electrically coupled to the electromagnetic-wave detection circuit for calculating and analyzing the reflection electromagnetic-wave signal to get diabetes factor results.

3. The apparatus for measurement of diabetes factors of claim 1, wherein the test sample is a human body fluid, including a saliva sample, sweat sample or tear sample.

4. The apparatus for measurement of diabetes factors of claim 1, wherein the diabetes factor results include an insulin concentration or a glucose concentration of the test sample.

5. A apparatus for measurement of diabetes factors of claim 1, wherein the display device is electrically or wirelessly coupled to the microcontroller for displaying measurement and analysis the diabetes factor results.

6. A apparatus for measurement of diabetes factors of claim 1, wherein the electromagnetic-wave signal generated by the miniature electromagnetic-wave generator is a millimeter wave.

7. A apparatus for measurement of diabetes factors of claim 6, wherein the millimeter wave generated by the miniature electromagnetic-wave generator is with frequency band in the range of 1-300 GHz.

8. A apparatus for measurement of diabetes factors of claim 6, wherein the millimeter wave generated by the miniature electromagnetic-wave generator is with frequency 60 GHz.

9. The apparatus for measurement of diabetes factors of claim 1, wherein the miniature electromagnetic-wave generator is installed on a chip of integrated circuits, or embedded in a packaged chip of integrated circuits

10. The apparatus for measurement of diabetes factors of claim 1, wherein the miniature electromagnetic-wave generator is applied to the field of 3C (Computer, Communications, Consumer-Electronics) home appliances, the field of communications devices, including smart phones, and the field of Internet of Things (IoT) and Artificial Intelligence (AI).

11. A apparatus for measurement of diabetes factors of claim 1, wherein the display device includes complete graphics display systems, portable electronics, laptop and desktop computers, television/video systems, liquid crystal displays, subtractive displays, plasma panel displays, electro-luminescence (EL) displays, electrophoretic displays, field emitter displays, discrete light emitting diode displays, organic light emitting diodes (OLEDs) displays, projectors, cathode ray tube (CRT) displays, and combinations comprising at least one of the foregoing displays.

12. A method for measurement of diabetes factors using the apparatus for measurement of diabetes factors as claim 1, at least comprising the steps of:
providing a characteristic curve;
providing a test sample on the sample preparation device;
starting the miniature electromagnetic-wave generator to generating the electromagnetic-wave signal;
sending the electromagnetic-wave signal from the miniature electromagnetic-wave generator to the test sample of a tester as a scanning signal to scan the test sample;
collecting the electromagnetic-wave signal reflected by the test sample after scanning the test sample to form a reflection electromagnetic-wave signal;
detecting the reflection electromagnetic-wave signal reflected from the test sample by the electromagnetic-wave detection circuit;
comparing the reflection electromagnetic-wave signal of the test sample of the tester with the characteristic curve for calculating a diabetes factor result corresponding to the test sample of the tester; and
outputting and displaying the diabetes factor results of the test sample of the tester by the display device.

13. The method for measurement of diabetes factors as claim 12, wherein the test sample is a human body fluid, including a saliva sample, sweat sample or tear sample.

14. The method for measurement of diabetes factors as claim 12, wherein the characteristic curve is depicted by measuring the diabetes factors of a standard saliva sample with known diabetes factor results as the horizontal axis, and corresponding reflection electromagnetic-wave signal as the vertical axis, wherein the diabetes factor results include an insulin concentration or a glucose concentration of the test sample.

15. The method for measurement of diabetes factors as claim 12, further comprising the step of confirming that the reflection electromagnetic-wave signal corresponds to the test sample of the tester by the microcontroller.

16. A system for measurement of diabetes factors comprising:
an apparatus for measurement of diabetes factors, comprising:
a host device including a miniature electromagnetic-wave generator; and
a sample preparation device,
wherein the host device is electrically or wirelessly coupled to the display device and the sample preparation device, and the miniature electromagnetic-wave generator is used for generating an electromagnetic-wave signal to scan a test sample on the sample preparation device; and
a communication device wirelessly connected with the apparatus for measurement of diabetes factors.

17. The system for measurement of diabetes factors of claim 16, wherein the host device further comprises:
a power supply for supplying power to the host device;
the miniature electromagnetic-wave generator electrically coupled to the power supply, for generating the electromagnetic-wave signal and transmitting the electromagnetic-wave signal to perform the scanning function on the test sample;
an electromagnetic-wave detection circuit electrically coupled to the miniature electromagnetic-wave generator, for detecting a reflection electromagnetic-wave signal from the test sample; and
a microcontroller electrically coupled to the electromagnetic-wave detection circuit for calculating and analyzing the reflection electromagnetic-wave signal.

18. The system for measurement of diabetes factors of claim 17, wherein the miniature electromagnetic-wave generator includes transmitters, the electromagnetic-wave detection circuit includes receivers, and the miniature electromagnetic-wave generator and the electromagnetic-wave detection circuit are arranged in a matrix pattern and enclosed in a compact hardware transceiver chip together.

19. The system for measurement of diabetes factors of claim 16, wherein the apparatus for measurement of diabetes factors further comprises: a display device electrically or wirelessly coupled to the host device, the display device set integrally within the host device or set on the communication device outside of the host device.

20. The system for measurement of diabetes factors of claim 16, further comprising a network server wirelessly connected with the apparatus for measurement of diabetes factors and the communication device respectively.
